(19) United States
(12) Patent Application Publication
(10) Pub. No.: US 2006/0253184 A1
(43) Pub. Date: Nov. 9, 2006

(54) System for the controlled delivery of stents and grafts

(76) Inventor: Kurt Amplatz, North Oaks, MN (US)

Correspondence Address: NIKOLAI & MERSEREAU, P.A., 900 SECOND AVENUE SOUTH, SUITE 820, MINNEAPOLIS, MN, 55402, US

(21) Appl. No.: 11/121,386

(22) Filed: May 4, 2005

(51) Int. Cl. A61F 2/06

(52) U.S. Cl. 623/1.11

(57) ABSTRACT

The present invention provides a delivery mechanism for percutaneously routing a stent or graft through the vascular system and procedures for addressing an aneurysm or an otherwise damaged vessel. The delivery system includes an outer tubular guide catheter 20, an inner tubular delivery (pusher) catheter 14 coaxially disposed and slidable relative to the outer guide catheter and an elongated flexible wire or cable 26 that is coaxially insertable through the lumen of the inner tubular catheter and that has a frusto-conical bead affixed at the distal end thereof which is sized to at least partially fit within the lumen of the inner pusher catheter when a proximally directed tension force is applied between the elongated flexible wire or cable 26 with respect to the pusher catheter 14. By inserting a compressed coil spring between a proximal end portion of the pusher catheter 14 and the proximal end portion of the cable 26, the requisite clamping force is maintained to secure the stent or graft to the distal end of the pusher catheter until the compression spring force is removed. With the stent or graft clamped to the distal end of the inner pusher catheter, it can be drawn within the lumen of the outer guide catheter for delivery therewith to the target site.

### BACKGROUND OF THE INVENTION

II. Field of the Invention

This invention relates generally to percutaneous transluminal vascular procedures and more particularly to delivery apparatus for placing a stent, a stent graft or a tubular graft at a desired target location within a subject's vascular system.

II. Discussion of the Prior Art

In the field of interventional cardiology, it is now becoming routine to treat stenotic lesions in the vascular system using balloon angioplasty to render more patent a partially occluded blood vessel and to attempt to thwart restenosis by placement of a stent at the site of the treated lesion.

Stents used in these procedures must be capable of assuming a reduced diameter configuration for delivery through a guide catheter, but which are either self-expanding upon exit of the distal end of the guide catheter or “balloon expandable”.

In carrying out a balloon angioplasty procedure with stenting, the Seldinger technique is frequently used to gain access to the vascular system and a tubular introducer having a hemostatic valve for preventing blood loss is inserted and typically, a puncture wound is made in the artery. A guide catheter is then inserted through the introducer and routed through the vascular system until the distal end portion of the guide catheter is disposed at an ostium of a selected artery having the stenotic lesion.

Next, an angioplasty catheter may be advanced over a guide wire sufficiently far so that an expandable balloon on the distal end of the delivery catheter is juxtaposed relative to the stenotic lesion. Upon inflation of the balloon, the stenotic lesion is compressed relative to the wall of the blood vessel being treated. If the balloon also carries a radially collapsed stent in surrounding relation to the balloon, as the balloon is expanded, so is the stent which becomes pressed against the vessel wall. Now, upon deflation of the balloon, it can be extracted leaving the stent in place.

Stents intended for use in percutaneous transluminal angioplasty applications come in various sizes depending on the vessel being treated.

Grafts are used for the treatment of aneurysms and commonly involve a tubular metal or polymeric scaffold having a fabric covering preventing blood leakage there through. Because of this construction, such grafts could not be compressed sufficiently to pass through an introducer like those used in executing the Seldinger procedure. As such, the medical team involved required a surgeon to perform a cut-down procedure.

Because of the radial size of most prior art vascular grafts of the covered scaffold variety typically would require a 24 Fr delivery sheath. Moreover, once the graft is delivered from the distal end of the delivery sheath, it is incapable of being retracted back into the sheath should repositioning be required.

What is needed, then, is an apparatus that will allow the controlled delivery if stents and grafts using percutaneous translumenal delivery thereby obviating the need for a surgeon. Further, a need exists for a delivery system for stents, stent grafts and grafts wherein the device to be delivered remains affixed to the delivery device, thus allowing the stent, stent graft or graft to be extended from and retracted into a delivery sheath repeatedly until such device is precisely positioned and deemed to be of the appropriate size to address the particular lesion or aneurysm involved. As used herein, a stent is a tubular scaffold for bridging a stenotic lesion in a blood vessel, a stent graft is a stent having a fabric, blood impervious covering and a graft is a scaffold for bridging a true aneurysm, a false aneurysm or a berry aneurysm. Such devices are collectively referred to herein as a vascular prosthesis or simply a prosthesis.

### SUMMARY OF THE INVENTION

The foregoing desired objects are achieved in accordance with the present invention by providing an apparatus for percutaneously delivering a self-expanding stent or graft to a target site within a patient's vascular system. The apparatus comprises an outer tubular guide catheter having a proximal end, a distal end and a lumen extending there between along with an inner tubular pusher catheter also having a proximal end, a distal end and a lumen and where the inner pusher catheter has an outer diameter sized to slidingly fit within the lumen of the guide catheter. An elongate, flexible member is coaxially inserted through the lumen of the inner pusher catheter and it has a first bead member affixed to its distal end where the bead is sized to at least partially fit within the lumen of the inner pusher catheter at the distal end of the pusher catheter when a proximally directed tension force is applied to the proximal end of the elongated flexible member with respect to the inner pusher catheter. Completing the apparatus is a compression spring that is operatively coupled between the proximal end of the inner pusher catheter and a clamp member that is releasably affixed to the elongate member near the proximal end of the elongate member.

The stent, stent graft or graft deployed using the apparatus of the present invention comprises a large plurality of very fine braided metal strands exhibiting a memory property and which is radially collapsible to a relatively small size for passage through the outer tubular guide catheter but which, when released from the guide catheter, self-expands to a relatively large diameter. The number of strands, the diameter of each strand, the pitch and pick of the braid are such that the pore size of the resulting tubular graft is sufficiently small that fibrin present in the blood will close such pores, rendering the graft leak-proof. The braided tubular graft is installed on the delivery system by capturing the free ends of the strands comprising the braided graft at its proximal end between the bead member affixed to the elongate flexible member and the wall defining the lumen of the inner tubular pusher catheter at its distal end. The compression spring is used to maintain the requisite tension force on the elongate member to maintain the ends of the strands pinched between the bead member and the wall of the inner tubular pusher catheter proximate its distal end.

### DESCRIPTION OF THE DRAWINGS

The foregoing features, objects and advantages of the invention will become apparent to those skilled in the art from the following detailed description of a preferred embodiment, especially when considered in conjunction with the accompanying drawings in which like numerals in the several views refer to corresponding parts.

FIG. 1 is a partial side elevation view illustrating the percutaneous delivery system for stents and grafts configured in accordance with the present invention;

FIG. 2 is a greatly enlarged view of the distal end portion of the assembly of FIG. 1 showing the proximal ends of the wires comprising the braided stent or graft captured at the distal end of the delivery catheter; and

FIG. 3 is a view like that of FIG. 2 showing the stent or graft released from the distal end of the delivery catheter.

### DESCRIPTION OF THE PREFERRED EMBODIMENT

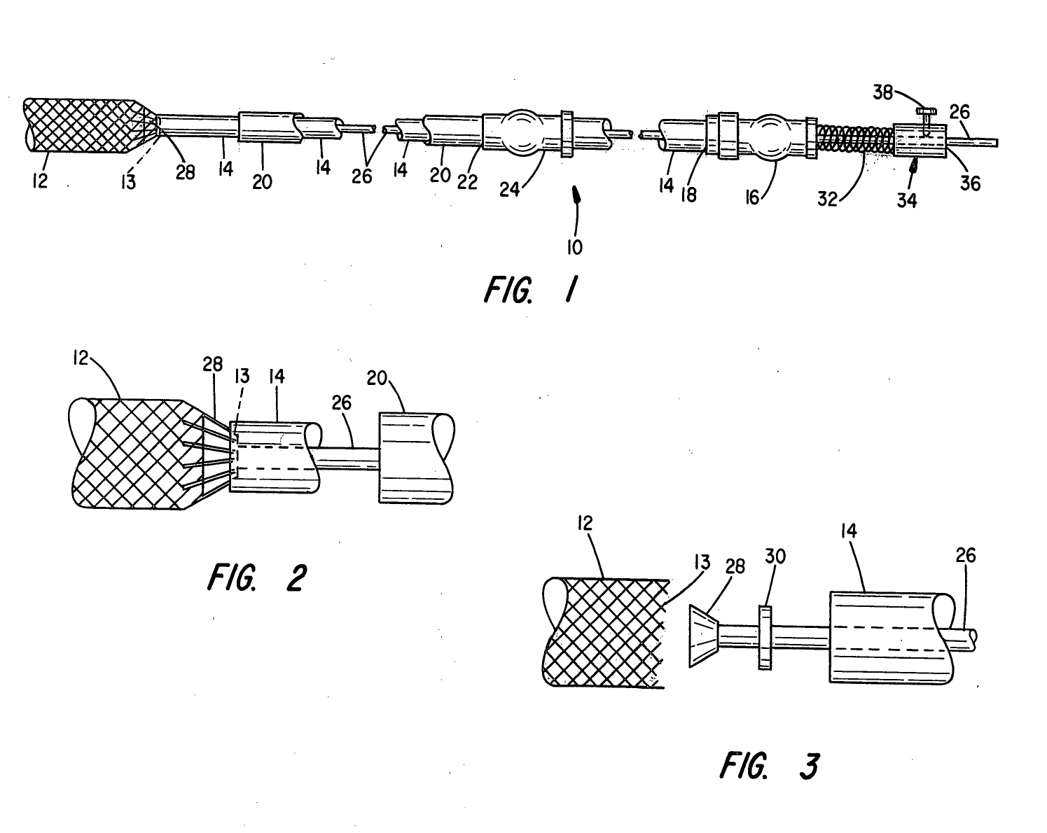

Referring first to FIG. 1, the percutaneous translumenal stent or graft delivery system is identified generally by numeral 10 and, as already indicated, is used to deliver a stent or graft member 12 to a target site within the vascular system such as at the location of an abdominal aortic aneurysm for the purpose of exclusion of the aneurysm to prevent further bulging and possible rupture thereof.

The vascular prosthesis 12 is preferably formed of a metal fabric exhibiting an expanded configuration and a collapsed configuration. The prosthesis, when collapsed, can be deployed through the lumen of a catheter and, upon exiting the distal end of the catheter at a target site in a patient's vascular system, will substantially return to its expanded configuration.

As is described in U.S. Pat. No. 5,725,552 to Curtis Amplatz, the metal fabric comprising the prosthesis may comprise a plurality of braided metal strands where the metal is preferably a shape memory alloy such as NITINOL®. In accordance with the present invention, the metal fabric is braided in the form of a tube that can be fitted onto a cylindrical mandrel and then heat-treated so that in its expanded configuration, the prosthesis will have an internal diameter substantially equal to the outer diameter of the mandrel on which it is heat-treated.

Without limitation, the graft may comprise a 72, a 144, or a 288-strand tubular wire braid using wires of selected diameters dependent on the number of wires employed in the braiding process Using a tubular braid of about 20-30 mm in diameter with a predetermined pitch and pick such that the graft exhibits a pore size less than 100 microns, the graft can be longitudinally stretched to a reduced diameter permitting it to be passed through the lumen of a 7 French guiding catheter that can readily be inserted into the vascular system using the Seldinger technique. Upon exit from the distal end of the delivery catheter at the desired target site, the graft 12 will self-expand to a limit defined by the vessel wall in which it is disposed.

Using a metal fabric braided from 288 or even 144 strands or wires whose diameters may be about 0.00075 inch yields a fabric that is rather blood-impervious and within a relatively short time following placement becomes endothelialized. Blood trapped between the outer surface of the graft and the bulge comprising the aneurysm rapidly clots to fill the bulge space with a congealed mass. The lumen of the graft, however, remains patent, allowing continuous blood flow through the treated area of the blood vessel.

Those skilled in the art interested in obtaining more information concerning the fabrication of occluding devices using braided structures of the type contemplated herein for the prosthesis 12 are referred to the following patents, each of which is assigned to AGA Medical Corporation, the assignee of the present application, the teachings of which are hereby incorporated by reference.

U.S. Pat. No. 5,725,552

U.S. Pat. No. 5,944,738

U.S. Pat. No. 6,468,303

U.S. Pat. No. 6,402,772

U.S. Pat. No. 6,468,301

U.S. Pat. No. 6,368,339

U.S. Pat. No. 6,506,204

Turning next to the construction of the graft delivery device 10, it is seen to comprise a pusher catheter 14 having a male Luer coupler 16 of a standard variety affixed to its proximal end 18. The delivery catheter may be of various lengths and may have an outer diameter of from about 50 to 10 French, depending on the location of the vessel segment to be treated, allowing it to pass through an internal lumen of an outer guide catheter 20.

The guide catheter 20 has a lumen of a size to receive the pusher catheter 14 therethrough with a close tolerance so that blood flow between the two is substantially blocked. Affixed to its proximal end 22 of the guide is a female Luer fitting 24 that is adapted to mate with the male Leur fitting 16 affixed to the proximal end 18 of the delivery catheter 14.

Disposed within the lumen of the pusher catheter 14 is a wire or cable 26 whose length allows it to extend beyond the full length of the delivery catheter 14 when pushed from its proximal end portion. Laser welded to the distal end of the cable or wire 26 is a bead that is spherical or frusto-conically shaped clamp member 28 and a short, predetermined distance proximal of the clamp member 28 is an annular washer-like member 30 that is also welded or otherwise fixedly attached to the cable or wire 26.

A helically-wound compression spring 32 slips over and surrounds the cable or wire 26 and is operatively disposed between the proximal end of the male Luer fitting 16 and a releasable clamp 34 here shown as a tubular sleeve 36 having a transversely extending threaded bore leading to the lumen of the tubular sleeve 36. Fitted into this threaded bore is a thumbscrew 38 that when tightened down against the wire or cable 26 serves to lock the sleeve 36 to that cable or wire.

To ready the delivery system for use, the free ends of the strands comprising the braid at the proximal end 13 fed into the lumen of the pusher catheter 14 and are captured between the outer surface of the bead member 28 and the distal end 15 of the pusher catheter 14, as best seen in the greatly enlarged partial view of FIG. 2. To achieve this result, the prosthesis 12 in its expanded configuration is slipped over the tapered clamp member 28 and the proximal end of the cable or wire 26 is fitted through a disposable, tear-away funnel member (not shown) before being inserted into the distal end 15 of the pusher catheter 14 and fed down its length. As the prosthesis is pushed through the funnel, the proximal ends of the strands are made to feed into the lumen of the pusher catheter 14 and now, as the cable or wire 26 is pulled in the proximal direction, the proximal ends of the wire strands 13 become captured between the bead member 28 and the lumen wall of the pusher catheter 14. So long as the tension is maintained, the free ends 13 of the braided prosthesis 12 will remain captured.

To maintain the prosthesis clamped to the distal end of the pusher catheter 14, tension is applied at the proximal end of the wire or cable 26 as the sleeve 36 is pushed in the distal direction to thereby compress the coil spring 32 between the sleeve 36 and the Luer fitting 16. With the spring 32 so compressed, the thumbscrew 38 will be tightened to thereby hold the sleeve 36 in position relative to the coil or wire 26, thus maintaining the tension force on the cable or wire 26.

The assembly comprising the pusher catheter 14, the compression spring 32, the clamping member 34 can be drawn in the proximal direction while holding the female Luer fitting 24 stationary, thus drawing the distal end 15 of the pusher catheter along with the prosthesis 12 into the lumen of the outer guide catheter 20. All of these steps of clamping the braided device to the pusher catheter 14 and drawing the prosthesis 12 within the lumen of the outer guiding catheter 20 may be performed at a manufacturer's facility prior to packaging and sterilization of the assembly. At the time of use with a patient, a cardiologist may first gain percutaneous entry of the guide catheter 20 containing the stent or a stent/graft or a graft (the prosthesis) and route the distal end thereof under fluoroscopic viewing to the target site of an aneurysm to be reinforced. While keeping the outer guide catheter 20 stationary, the pusher catheter 14 is advanced in the distal direction until its distal end 15 to which the prosthesis 12 is clamped exits the distal end of the guide catheter 20. So long as the compression spring is providing the tension force on the cable, the prosthesis remains coupled to the distal end of the pusher catheter allowing it to be again retracted into the lumen of the outer guide catheter should it become necessary to reposition the device before it is released.

To release the prosthesis from the distal end 15 of the pusher catheter 14, the physician merely has to loosen the thumbscrew 38 and then move the cable or wire 26 in the distal direction sufficiently far so that the washer 30 pushes against the proximal end surfaces of the wires 13 to move the prosthesis free of the end of the pusher catheter. At this point, and as shown in FIG. 3, the prosthesis 12 has self-expanded to a larger diameter so that the bead 28 can readily be withdrawn from the interior of the tubular prosthesis. The delivery system 10 can then be withdrawn from the vascular system.

While a preferred embodiment of the present invention has been described, it should be understood that various changes, adaptations and modifications may be made therein without departing from the spirit of the invention and the scope of the appended claims. For example rather than front loading the pusher catheter 14 carrying the elongate member 26 and the prosthesis 12 by feeding the proximal end of the pusher catheter through the distal end of the delivery sheath 20 and then along the length of the delivery sheath, it is also contemplated that a loader tube containing the prosthesis be coupled to the Luer fitting 24 and the pusher wire 26 be used to advance the prosthesis down the delivery sheath until it approaches the distal end of the delivery sheath 20.

I claim:

1. Apparatus for percutaneously delivering one of a stent, stent draft or graft prosthesis to a target site within a patient's vascular system comprising:
(a) an outer tubular guide catheter having a proximal end, a distal end and a lumen extending therebetween;
(b) an inner tubular pusher catheter having a proximal end, a distal end and lumen extending therebetween, the inner pusher catheter having an outer diameter sized to slidingly fit within the lumen of the guide catheter;
(c) an elongate, flexible member coaxially insertable through the lumen of the inner pusher catheter, said elongate, flexible member having a proximal end and a distal end, said distal end having a first bead member affixed thereto sized to at least partially fit within the lumen of the inner pusher catheter at said distal end of the inner pusher catheter when a proximally directed tension force is applied to the elongate flexible member with respect to the inner pusher catheter; and
(d) a compression spring operatively coupled between the proximal end of the inner pusher catheter and a clamp member releasably affixed to the elongate member.

2. The apparatus as in claim 1 wherein the elongate member further includes a second bead member affixed thereto at a location a predetermined distance proximal of the first bead member, the second bead member sized to slidingly fit within the lumen of the inner tubular catheter.

3. The apparatus as in claim 1 wherein the first bead member is frusto-conically shaped.

4. The apparatus in claim 1 wherein the first bead member is spherical.

5. The apparatus as in claim 2 wherein the second bead member comprises an annulus.

6. The apparatus as in claim 3 wherein a proximal end portion of the self-expanding prosthesis is captured between the frusto-conical shaped bead and a wall defining the lumen of the inner pusher catheter at the distal end thereof when the tension force is being applied.

7. The apparatus as in claim 1 wherein the elongate, flexible member comprises a wire.

8. The apparatus as in claim 1 wherein the elongate, flexible member comprises a cable.

9. The apparatus as in claim 1 wherein the outer tubular guide catheter includes a first Luer fitting on the proximal end thereof and the inner pusher catheter includes a second Luer fitting on the proximal end thereof for mating with the first Luer fitting.

10. The apparatus as in claim 7 wherein the compression spring is disposed in surrounding relation with respect to the elongate member between the second Luer fitting and the clamp member.

11. The apparatus as in claim 3 wherein the self-expanding prosthesis is released from the distal end of the inner catheter when the compression spring is no longer under compression.

12. The apparatus as in claim 1 wherein the self-expanding prosthesis comprises a plurality of fine metal wire strands interwoven to form a tubular metal fabric having open proximal and distal ends and where the metal strands at the proximal end of the tubular metal fabric are adapted to be captured between the first bead member and a wall defining the lumen of the inner pusher catheter at the distal end thereof when said tension force is applied.

13. The apparatus as in claim 12 wherein the fine wires comprise shape memory alloy wires.

14. The apparatus as in claim 13 wherein the shape memory alloy is Nitinol.

15. Apparatus for percutaneously delivering a self-expanding prosthesis to a target site within a patient's vascular system comprising:
(a) a tubular inner catheter having a proximal end, a distal end and lumen extending therebetween, the inner catheter having an outer diameter adapted to slidingly fit within the lumen of a guide catheter;
(b) an elongate, flexible member coaxially insertable through the lumen of the inner tubular catheter, said elongate, flexible member having a proximal end and a distal end, said distal end having a first bead member affixed thereto sized to at least partially fit within the lumen of the inner tubular catheter at said distal end of the inner tubular catheter when a proximally directed tension force is applied to the elongate flexible member with respect to the inner tubular catheter; and
(c) a compression spring operatively coupled between the proximal end of the inner tubular catheter and a clamp member releasably affixed to the elongate member.

16. The apparatus as in claim 15 wherein the elongate member further includes a second bead member affixed thereto at a location a predetermined distance proximal of the first bead member, the second bead member sized to slidingly fit within the lumen of the tubular catheter.

17. The apparatus as in claim 15 wherein the first bead member is frusto-conically shaped.

18. The apparatus as in claim 15 wherein the first bead member is spherical.

19. The apparatus as in claim 16 wherein the second bead member comprises an annulus.

20. The apparatus as in claim 17 wherein a proximal end portion of the self-expanding prosthesis is captured between the first bead member and a wall defining the lumen of the inner tubular catheter at the distal end thereof when the tension force is being applied.

21. The apparatus as in claim 15 wherein the elongate, flexible member is a wire.

22. The apparatus as in claim 15 wherein the elongate, flexible member comprises a cable.

23. The apparatus as in claim 21 wherein the compression spring is disposed in surrounding relation with respect to the elongate member between the second Luer fitting and the clamp member.

24. The apparatus as in claim 17 wherein the self-expanding prosthesis is released from the distal end of the inner tubular catheter when the compression spring is no longer under compression.

25. The apparatus as in claim 15 wherein the self-expanding prosthesis comprises a plurality of fine metal wire strands interwoven to form a tubular metal fabric having open proximal and distal ends and where the metal wire strands at the proximal end of the tubular metal fabric are adapted to be captured between the first bead member and a wall defining the lumen of the inner tubular catheter at the distal end thereof when said tension force is applied.

26. The apparatus as in claim 25 wherein the fine wires comprise shape memory alloy wires.

27. The apparatus as in claim 25 wherein the shape memory alloy is Nitinol.

28. A method for controllably delivering a braided self-expanding tubular prosthesis to a selected site in the vascular system comprising the steps of:
(a) providing the combination of:
(i) a flexible tubular inner catheter having a proximal end, a distal end and a lumen extending therebetween, the inner catheter having an outer diameter adapted to slidingly fit within the lumen of a guide catheter,
(ii) an elongate, flexible member coaxially insertable through the lumen of the inner tubular catheter, said elongate flexible member having a proximal end and a distal end, said distal end having a first bead member affixed thereto sized to at least partially fit within the lumen of the inner tubular catheter at said distal end of the inner tubular catheter when a proximally directed tension force is applied to the elongate flexible member with respect to the inner tubular catheter,
(iii) a compression spring operatively coupled between the proximal end of the inner tubular catheter and a clamp member releasably affixed to the elongate member; and
(b) a braided tubular prosthesis with the proximal ends of individual strands comprising the prosthesis captured between the first bead member and the distal end of the inner tubular catheter;
(c) feeding the inner tubular catheter with the braided tubular prosthesis attached through a lumen of the guide catheter and out a distal end thereof;
(d) releasing the clamp member; and
(e) advancing the elongate flexible member in a distal direction to release the tubular prosthesis from the distal end of the inner catheter.
